(19) United States
(12) Patent Application Publication
(10) Pub. No.: US 2012/0231964 A1
(43) Pub. Date: Sep. 13, 2012

(54) METHOD FOR SELECTING NUCLEIC ACIDS THAT BOND WITH HIGH-AFFINITY TO A TARGET

(75) Inventor: Andreas Kage, Berlin (DE)

(73) Assignee: APTARES AG, Luckenwalde (DE)

(21) Appl. No.: 13/270,325

(22) Filed: Oct. 11, 2011

Related U.S. Application Data
(63) Continuation of application No. 12/262,076, filed on Oct. 30, 2008, now abandoned.
Application No. 12/262,076 is a continuation of application No. 10/398,469, filed on Oct. 29, 2003, now abandoned.

(30) Foreign Application Priority Data
Oct. 2, 2000 (DE) 10049074.3

(51) Int. Cl. C40B 30/04; C40B 40/06; C07H 21/04

(52) U.S. Cl. 506/9; 536/24.3; 506/16

(57) ABSTRACT

The invention relates to a method for selecting nucleic acids that bond with high affinity to a target molecule from a mixture of nucleic acids, comprising: a) loading a column with the target molecules, b) feeding the nucleic acids into a first end of the column, to create a defined volumetric flow of medium through the column, c) immobilizing the nucleic acids to the target molecule wherein an affinity of the nucleic acids to the target molecule decreases as the distance from the first end of the column increases, d) stopping the volumetric flow after a defined period of time, e) cutting the column into segments, and allocating a routing co-ordinate to each segment, and f) identifying and collecting nucleic acids that bond with a high affinity to the target molecule by desorbing the immobilized nucleic acids from at least one segment.

### STATEMENT OF RELATED APPLICATIONS

This is a continuation of U.S. patent application Ser. No. 12/262,076, filed Oct. 30, 2008, entitled METHOD FOR SELECTING NUCLEIC ACIDS THAT BOND WITH HIGH-AFFINITY TO A TARGET, which is a continuation of U.S. patent application Ser. No. 10/398,469, filed Oct. 29, 2003, entitled METHOD FOR SELECTING NUCLEIC ACIDS THAT BOND WITH HIGH-AFFINITY TO A TARGET, which is a 371 of PCT International Patent Application PCT/DE01/03817, filed Oct. 2, 2001, entitled METHOD FOR SELECTING NUCLEIC ACIDS THAT BOND WITH HIGH-AFFINITY TO A TARGET, which claims the benefit of priority of German Patent Application No. DE10049074.3, filed Oct. 2, 2000. Each of the prior applications is incorporated by reference herein in its entirety.

### FIELD OF THE INVENTION

The invention relates to a method for selecting nucleic acids that bond with high affinity to a target, wherein a mixture of nucleic acids is contacted with one or several defined target molecules, wherein nucleic acids bonding to the target molecule are immobilized, and wherein the nucleic acids bonded to the target molecule are desorbed by the target molecules after removal of not bonded nucleic acids.

Nucleic acids are poly or oligonucleotides with a nucleotide count of 5 to 200, in particular 20 to 200. These may be DNAs, RNAs or PNAs. In particular, the nucleic acids may be chemically derivatized, for instance by 2′ and/or 5 substitution, and/or provided with reporter molecules (molecules which permit a detection with conventional detection methods). The nucleic acid may be single or double-stranded. A target molecule may in principle by of any type, as far as it is not such a nucleic acid which enters into Crick/Watson base pair bonds with the nucleic acid contacted therewith. Examples for target molecules are: peptides, proteins, enzymes, oligosaccharides, polysaccha-rides, nucleic acids not entering into Crick/Watson bonds, lipids, but also hormones and other organic compounds (pheromones). Targets may also be parts of cells and complete cells, same as complete viruses and bacteria. Nucleic acids binding to non-nucleic acids are designated as aptamers, but also nucleic acids entering into non-Crick/Watson bonds with other nucleic acids. The last-mentioned aptamers may for instance be used for the detection of certain gene defects and/or deletions in genes. The term bonds means in this invention noncovalent bonds. The term affinity relates in this invention to the binding force within complexes of the antigen/antibody type. The binding force is quantifiable by the affinity constant, which is defined under the law of mass action. In this invention, the term affinity however not only relates to the complexes with a binding site, but also to complexes having several binding sites, i.e. also comprises the term avidity. The avidity results from the number and the binding force of every binding site for multivalent antibody/antigen complexes. As an amplification is understood every enzymatically mediated reaction serving for the replication of a nucleic acid, for instance the PCR.

### BACKGROUND OF THE INVENTION AND PRIOR ART

Nucleic acids serve in natural organisms mainly for coding proteins to be expressed in a cells and the like. This is determined by the primary structure of the nucleic acid, i.e. the sequence. Independent herefrom, antibody/antigen complexes may however also enter into bindings with non-nucleic acids existing in a cell or in special cases non-Crick/Watson bonds with other nucleic acids. Whether such a binding may occur, depends not only from the primary structure, but also from the secondary, tertiary and quaternary structure generated in a solution for a defined sequence (three-dimensional structure). The affinity of the nucleic acid to the target molecule at last depends from whether the nucleic acid—in addition to the purely chemical binding capability—“matches” in steric regard in the region of the binding site or the binding sites of the target molecule, corresponding to the conditions for classic antibody/antigen complexes. Matching nucleic acids may thus exert the function of an antibody or antigen. Such aptamers normally are non-natural nucleic acids and can so to speak “tailored” for a target molecule. For tailoring, there are in principle two approaches. The first approach is the calculation of a suitable sequence and/or derivatization for the nucleic acid according to the precisely known structure, including binding sites and tertiary structure, of the target molecule. This is not only extremely costly; in cases where the structure of the target molecule is not sufficiently known, this approach is even impossible. The second approach consists in the isolation of the target molecule and in the contacting of a mixture of prospective nucleic acids with the isolated (and in most cases immobilized) target molecule, wherein nucleic acids that bond with high affinity are separated from those that bond with less affinity or do not bond at all. The mixtures of the nucleic acids typically are nucleic acid libraries for instance established by the combinatorial chemistry. A nucleic acid library contains a plurality of different nucleic acids, at least in a partial sequence region a randomization (with natural and/or non-natural nucleotides) is provided. A preserved sequence region may be provided, is however not necessary. Randomization in n positions with m different nucleotides leads to a library with nm elements. The selected high-affinity nucleic acids are suitable for a plurality of applications.

For instance, nucleic acids may be used in tests for the existence or non-existence of target molecules specific for the nucleic acid in a test solution and/or in a cell or tissue. Then the presence of a reporter molecule of a conventional structure in the nucleic acid for the easy identifiability by a measurement is recommended. Such tests may be used in the diagnostics, for instance the diagnostics for oncogenic mutants or marker substances resulting from certain physiological malfunctions. It is understood that the respective nucleic acid needs not only selectively “detect” the oncogenic mutant, but must not bind to the natural variant, i.e. must discriminate between an oncogenic expression product and a natural expression product. This can easily be performed after the selection of nucleic acids bonding with the oncogenic expression product, namely by the subsequent selection of nucleic acids which do not bond with the natural expression product from the previously selected nucleic acids.

Selected nucleic acids may also be used for the separation of target molecules from a solution, for instance in conventional column or gel separation methods. The it is recommended to have the nucleic acid immobilizable and to immobilize it in the separation method.

Selected nucleic acids may however also be used for modulating physiological functions, i.e. inhibiting, inducing or reinforcing. Such nucleic acids are thus also used in pharmaceutical compositions. In addition, selected nucleic acids have of course to be physiologically acceptable in order to avoid side effects. The advantage of using nucleic acids in lieu of for instance peptides is that the identification or selection of suitable nucleic acids is considerably easier than in the case of the peptides or proteins because of the comparatively easy producibility with regard to protein or peptide libraries.

A known method for selecting nucleic acids that bond with high affinity to a target molecule is known as the SELEX method (Systematic Evolution of Ligands by EXponential enrichment). Various variants are for instance described in the documents U.S. Pat. No. 5,712,375, U.S. Pat. No. 5,864,026, U.S. Pat. No. 5,789,157, U.S. Pat. No. 5,475,096, U.S. Pat. No. 5,861,254, U.S. Pat. No. 5,595,877, U.S. Pat. No. 5,817,785. In the insofar known methods, in principle approximatively in a plurality of cycles such nucleic acids are separated which bond with high or higher and higher affinity. In every cycle, the selected group must be amplified with nucleic acids. The separation of bonding nucleic acids from the target in every cycle takes place by specific driving-out. This method has several drawbacks. First of all, it is disadvantageous that due to the required number of cycles a rather large amount of nucleic acids as well as of target molecules is necessary. Further, in the driving-out step, more (bonded) nucleic acids of lower affinity are driven out from the bond that nucleic acids of higher affinity, thus the difference in amounts is increased at the expense of the higher-affinity nucleic acids in the amplification step. The difference in amounts is further increased by that with higher affinity the bond of the separated nucleic acids to the ligands used for the separation is comparatively stronger, and the higher-affinity nucleic acids are thus less accessible for the amplification. It is further disadvantageous that with increasing affinity of the nucleic acids a logarithmically increasing concentration of the ligand used for the separation is required. The obtainable affinity is thus limited by the solubility product of the used ligands. Finally it is disadvantageous to have to operate in several cycles for the repeated separation or selection of nucleic acids selected in a pre-cycle.

In the field of the separation of non-nucleic acids, the affinity chromatography, in particular as column chromatography (solid/liquid phase) is well known. It is a method for the isolation or enrichment, up to 105 times and more, of for instance proteins. A ligand of the sequence to be enriched is immobilized at a chromatographic matrix. Highly affinitive compounds are firstly bound, i.e. at the entrance of the column. Downstream are bound less affinitive compounds, as far as the amount of the affinitive compounds, referred to the respective specific compound, is lower than the amount of ligands in the column. Weakly affinitive or not affinitive compounds will pass through and are thus separated from the affinitive compounds. Bound, i.e. affinitive compounds are then eluted and further used. With non-specific desorption methods, for instance physico-chemical or thermal methods, a mixture of differently highly affinitive (bound) compounds takes place. In the desorption with ligands of the bound compounds (driving-out desorption), a very high molar amount of the ligand is necessary for the separation in particular of the highly affinitive compounds.

### TECHNICAL OBJECT OF THE INVENTION

The invention is based on the technical object to provide a method for selecting nucleic acids that bond with high affinity to a target molecule, which supplies with less expenses highly affinitive nucleic acids having a very low variety in the selected nucleic acids.

### SUMMARY OF THE INVENTION

For achieving the technical object, the invention teaches a method for selecting nucleic acids that bond with high affinity to a target molecule from a mixture of nucleic acids, comprising the following steps: a) the interior of a column is loaded with the target molecules and the target molecules are immobilized in said column, b) the mixture of nucleic acids is fed into a first end of the column, to create a defined volumetric flow of medium through the column, running from the first end to the second end of said column, c) the nucleic acids bond with an affinity to the target molecule that decreases as the distance from the first end of the column increases and are immobilized, d) the volumetric flow of medium through the column is stopped after a defined period, e) the column is divided into column segments by a plurality of partitions, a routing co-ordinate being allocated to each segment, f) the immobilized nucleic acids from at least one segment are desorbed in a non-specific manner and are extracted using the allocation of the routing co-ordinate allocated to the segment. The term interior of the column means within a lumen in all generality. The term column comprises for the purpose of this invention all kinds of solid carrier systems, i.e. carrier systems not being completely enclosed are also possible. For the purpose of the invention, one column segments only may be provided as an extreme.

The immobilization of the target molecules may be performed according to the conventional methods of the column chromatography. One species of target molecules may be used, however several different defined species in a homogeneous distribution or sorted to the routing co-ordinate may be used within the column. The kind of immobilization depends in the individual case from the structure and/or the properties of the target molecule and can be selected easily by the man skilled in the art from the prior art. A column is every technical construct having a lumen with two ends. A structural material, all materials usual for columns can be used, such as metals, glass and/or plastic materials. The interior of the column may be provided with a matrix binding the target molecule, and/or the structural material may be suitable or prepared for a direct binding of the target molecules. A mixture of nucleic acids means nucleic acid libraries with a number of typically 106 to 1022/mole, in particular 1011 to 1021/mole, of different nucleic acid species. In the library applied on the column, each nucleic acid species is statistically present with for instance 10 to 1017, in particular 100 to 1013, molecules. The medium is normally a liquid wherein the nucleic acid library is soluble and stable. For this purpose can be used all buffers usual for nucleic acid libraries and the like. The volumetric flow of medium can be adjusted before the application of the nucleic acid library. Then the nucleic acid library is added at the entry of the column to the medium flow. The nucleic acid library may however also be applied immediately. After a period of time being determined by the design of the column and the volumetric flow, a “clot” applied by the nucleic acid library will leave at the exit of the column (widened by folding with the diffusion), bound nucleic acids being separated from the “clot” and immobilized in the column. Suitably, the volumetric flow through the column is adjusted to low or non-turbulence, preferably laminarity (sum of the acceleration vectors of the medium over the column volume, in particular over the column cross-section, is minimum, ideally 0). The total number of target molecules (of a target molecule species) in the column is typically the 102 to 1016-fold, in particular the 103 to 1015-fold, of the number of nucleic acid molecules of a single species in the applied nucleic acid library. The bonding of the nucleic acids to the target molecules preferably takes place under conditions that correspond to a later use of the nucleic acids, i.e. for instance in a buffer or a medium being correspondingly adjusted with regard to temperature, ionic strength, pH value and buffer conditions. The medium and the solvent of the nucleic acid library have then to be selected correspondingly according to the components thereof. The division of the column into a plurality of column segments may for instance be made by cutting the column into pieces, the cuts preferably being made orthogonally to the volumetric flow vector. The column may however also have previously been composed of column segments, a column segment densely lining-up preferably in the direction of the volumetric flow vector with the next column segment (joint cross section orthogonally to the volumetric flow vector). Then the division may be effected by dissociation of the previously formed assembly of column segments. The non-specific desorption may be performed in a physico-chemical or thermal manner by elution with a sufficiently strong ligand by driving-out, chelation, modification and/or destruction of the target molecule. Mechanical methods, for instance ultrasonic methods, may be used for the desorption or to the increase of the desorption. Combinations of the above desorption methods may also be used. It is understood that the nucleic acids must not be decomposed by the applied desorption methods.

The invention is based on the finding that a nucleic acid library can spatially be separated like a protein mixture by the affinity chromatography according to the affinity to the target molecule. The invention is further based on the finding that a mixture of desorbing nucleic acids of different affinity, said mixture occurring by means of non-specific desorption, can be avoided by that prior to the non-specific desorption, the column with the nucleic acids bound therein is divided so to speak into affinity sections, and the nucleic acids bound in the thus obtained affinity sections or column segments can be desorbed easily and without disturbing ligand couplings for instance by a PCR or in RT-PCR non-specific manner and amplified equally non-specifically. Subsequent seletion artifacts at the expense of higher-affinitive nucleic acids are avoided. Ligands, in particular high concentrations of ligands, are not required for the desorption. Finally, virtually all bound and then desorbed nucleic acid molecules are available for an amplification. This permits to use low nucleic acid concentrations. In principle it is already sufficient if each species is present in the nucleic acid library by one molecule in a statistical average. If the number of the target molecules in a column segment is statistically 1, then even individual nucleic acid species can be separated according to their affinity to the target molecule.

A special advantage of the invention is explained in the following. The separation of the nucleic acids according to the affinity leads to that nucleic acids in a column segment have a similar affinity with different specificity (different regions of the target are bound or are binding sites for the nucleic acids). Thus obtained nucleic acid mixtures may be used in applications which are reserved until today to oligoclonal antisera. For instance, a quantitative determination of target molecules is possible by using the nucleic acid mixture in a binding kit. The nucleic acid mixture of one column segment may be used for the specific immobilization of certain target molecules (capture property) and/or for the specific detection of the immobilized target molecules.

Nucleic acids isolated or nucleic acid mixtures produced with the method according to the invention (to be brief, nucleic acids) can be used in various ways. For a respective specific application it is only necessary to immobilize target molecules selected according to the application in the column. It is for instance possible to identify marker substances being characteristic for a disease, to determine with the method according to the invention nucleic acids that bond with high affinity thereto, and to use the thus selected nucleic acids as a main component of a test kit for the investigation for the marker substance or for the presence or the risk of the disease. Such test kits may of course also be used for the therapy control. The nucleic acids may also be used for preparing pharmaceutical compositions, for instance when an inhibition of the marker substance leads to a reduction or prevention of the symptoms. It is also possible, by selection of the target molecules, to select nucleic acids and use them as a pharmaceutical composition, which stimulate the generation of substances missing in the case of a disease in an organism. By selection of suitable target molecules, for instance nucleic acids may also be found which influence as effective substances the differentiation and/or stimulation or suppression of isolated cells (for instance blood cells, such as T cells, granulocytes, monocytes or thrombocytes). The same may be effected for cells in an aggregate (tissue) and the like in the field of tissue engineering. Finally, nucleic acids selected according to the invention may for instance be used in an affinity matrix as an immobilized phase (for instance pheresis technologies) or for the specific desorption of substances of an affinity matrix.

### DETAILED DESCRIPTION

In principle it is sufficient that a segment to which is allocated a desired affinity is (isolatedly) processed to the desorption. This requires however—except for a maximum affinity, when the (referred to the volumetric flow vector) first column segment is processed—an idea about the affinity distribution in the applied nucleic acid library. It is therefore normally preferred that the immobilized nucleic acids from each segment are separately desorbed and obtained under respective allocation of the routing co-ordinate of each segment to the nucleic acids obtained therefrom.

In principle, any kind of desorption is possible. Preferably, the non-specific desorption is performed by means of usual physico-chemical or thermal methods. Thermal desorption is effected by heating the column segment or the solution contained therein. Heating may for instance by made by electrical heating or irradiation of microwaves or IR. In particular, the heating technologies of the PCR technology are suitable. In addition to the amplification of nucleic acids or aptamers by means of polymerase, other amplification methods, for instance by means of ligase, may of course also be used. The non-specific desorption may be supported by a chemical modification of the target, e.g. oxidation by sodium periodate or the like, or by non-specific complexing, for instance by means of borate or the like for blocking cis-trans diol bonds in hydrocarbons.

Therefore it is preferred that the non-specific desorption is performed by a thermal desorption in a preferably extended high-temperature phase of a PCR or RT-PCR. Hereby a synergy effect is achieved, since normally, in particular when working with nucleic acid libraries of low concentrations of the nucleic acid species, an amplification is anyway required. For increasing the yield, for instance 5 to 60, preferably 20 to 60, most preferably 45 to 55 cycles are used. For the amplification, at least one marked primer can be employed. The primer may comprise at least one endonuclease interface. Such an interface serves for instance for freeing the amplificate from larger regions of the primer sequence. Nucleotide components in the primer or in the nucleic acids to be selected may be marked for instance by fluorescence dyes. As fluorescence dyes are mentioned for instance: Alexa® Fluor 488, FLUOR 532, Fluor 546, Fluor 568, Fluor 594, Oregon Green 488, Fluorescein, Rhodamine 6G, tetramethylrhodamine, Rhodamine B and Texas Red. The amplificate may also be marked at different ends by two different chemical modifications, if the groups introduced by the modification can be bound as ligands respectively at a different affinity matrix.

It is recommended, for a safe separation of non-affinitive and low-affinitive nucleic acids, to include washing steps between suitable steps of the method. In particular, it is preferred that between step d) and e) at least one washing step is performed. For washing, for instance the solvent or the substance of the nucleic acid library or the medium of the column is suitable.

It is preferred that the inner-side occupancy of the column with target molecules and the immobilization thereof is performed by means of covalent binding, preferably after activation with chemically highly reactive groups (for instance tresyl chloride, cyan bromide and/or periodate) or by bifunctional spacer compounds after modification with chemically less reactive groups (for instance amin, hydroxy, keto and/or carboxyl). Examples for the spacer structures of suitable spacer compounds are: substituted and unsubstituted C2-C10 alkyl groups, substituted and unsubstituted C2-C10 alkenyl groups, substituted and unsubstituted C2-C10 alkynyl groups, substituted and unsubstituted C4-C7 carbocyclic alkyl groups, substituted and unsubstituted C4-C7 carbocyclic alkenyl groups, substituted or unsubstituted C7-C14 aralkyl groups, a heterocyclic molecule with heteroatoms selected from nitrogen, oxygen, sulfur, said substitutions consisting of alkyl, alkenyl, alkynyl, alkoxy, thiol, thioalkoxy, hydroxyl, aryl, benzyl, phenyl, nitro, halogen, ether groups with 2 to 10 carbon atoms and 1 to 4 oxygen or sulfur atoms, polyalkyl glycol, halogen, hydroxyl, thiol, keto, carboxyl, amides, ether compounds, thioether, arnidine derivatives, guanidine derivatives, glutamyl derivatives, nitrate (ONO2), nitro (NO2), nitrile, trifluoromethyl (—CF3), trifluoromethoxy (—OCF3), O alkyl, S alkyl, NH alkyl, N dialkyl, O aralkyl, S aralkyl, NH aralkyl, amino, azido (N3), hydrazino (NHNH2), hydroxylamino (ONH2), sulfoxide (SO), sulfone (SO2), sulfide (S—), disulfide (S—S), silyl. Spacer compounds are typically bifunctional, wherein the functionalities can be equal or different and for instance selected from the group consisting of “N-hydroxysuccinimide and hydrazides”.

For reducing the variety within the nucleic acids obtainable from a column segment, it is preferred that each column segment contains in a statistical average 0.1 to 103, preferably 1 to 102, most preferably 1 to 10 target molecules. Adjusted thereto, the nucleic acid library may, as applied, contain in a statistical average 0.1 to 103, preferably 1 to 102, most preferably 1 to 10 nucleic acid molecules of one species.

For the structural material of the column, in principle all materials known for instance from the affinity chromatography may be used. Hereto belong column of silica gel or polymers, such as polyethylene after activation by chemical derivatization or plasma activation. The length of the column segments is suitably in the range from 0.1 μm to 1 mm, preferably 0.1 to 100 μm, most preferably 0.5 to 10 μm. Such sections can easily be made by means of a microtome. The inner diameter of the column is suitably in the range from 0.05 to 1 mm, preferably 0.1 to 0.5 mm, most preferably 0.2 to 0.4 mm.

It is suitable that prior to the actual separation (binding or mechanical dissociation) of the nucleic acids, undesired nucleic acids are eliminated. Undesired nucleic acids are for instance nucleic acids that bond to target molecule-free inner surfaces of the column. Then a target molecule-free column can be arranged upstream, and the nucleic acid library can before be conducted therethrough. Undesired nucleic acids may be such ones that are not intended to bond to certain alternative target molecules, i.e. discriminate (for instance do not bond to natural variants of oncogenic mutants). Then a column comprising the alternative target molecules can be arranged upstream. Undesired nucleic acids may also be such one that bond to one of several binding domains of a target molecule only. Then a column comprising target molecules wherein one or several binding domains are blocked can be arranged upstream.

In the above methods, a continuous, i.e. for instance by arranging columns in series, or discontinuous, for instance by an intermediate collection of the eluent from the previous column, operation may be performed.

For achieving another improvement of the affinity separation, in principle various ways can be employed. For instance, the nucleic acids desorbed from one or several segments may, if applicable after amplification, repeatedly be subjected to the method according to the invention, and/or the elutropia of the conditions for the desorption may be increased (temperature, ionic strength, pH value, buffer conditions). Alternatively, the spatial density of the target molecules and thus of the bound nucleic acids, i.e. the number of the target molecules in a column segment, may be reduced.

If a homogeneous distribution of several target molecule species in a column is used, the invention can be improved in various ways. It is possible that nucleic acids of equal or similar affinity, i.e. from one column segment, are separated, after the desorption and amplification after binding to the various target molecule species, by separation of the target molecule species, for instance by electrophoresis or chromatography. The binding of the nucleic acids may take place before or after the separation of the target molecules. Nucleic acids bound in this re-separation may then be freed or desorbed in all ways explained above in other connections from the target molecules. A corresponding approach may be selected, if a target molecule comprises several different binding sites and the nucleic acids are small relative to the target molecule.

### Example 1

### Determining the Sensitivity of a Column

Let's consider a rectangular target molecule of the dimensions 100 nm by 10 nm by 10 nm, and a binding to the structural material of the column with a main face, then the target molecule occupies 10-16 m2. A lumen of 1 mm diameter and 1 mm length has an inner surface of 3×10−6 m2. With an occupation degree of 0.0001 to 0.001 as obtainable in the column technology, a number of 105 to 106 target molecules per mm column results. By cutting the column into 1 μm column segments by means of a microtome, thus a number of approx. 102 to 103 target molecules per column segment are obtained, thus an extremely high sensitivity of the affinity separation with the application of usual nucleic acid libraries being achievable.

### Example 2

### Activating a Silica Gel Column with Tresyl Chloride

The column is rinsed with acetone. For the activation, a water-free solution (2 ml acetone, 1 ml tresyl chloride, some drops pyridine) was passed through the column (10 times column volume), and incubated over night on ice. Then, the column was rinsed with 20 times the column volume of 100% acetone (water-free). The activated column can be kept in 1 mM HCl.

### Example 3

### Activating a Polyethylene Column

A polyethylene hose is rinsed with 20 times the column volume at room temperature with a solution (2% potassium permanganate (KMnO4) (w/v) in concentrated sulfuric acid (H2SO4)) and then with distilled water. For a further coupling of the column surface, bi or polyvalent molecules may be used for crosslinking which have at least one reactive aldehyde group (for instance 1% glutaric aldehyde). These were passed for 1 h at 4° C. through the column. Thereafter, the reaction is stabilized by reducing conditions (e.g. by sodium cyano boron hydride (0.00025% w/v in 0.15 M NaCl, pH 3.9).

### Example 4

### Coupling a Protein to the Activated Silica Gel Column

The tresyl chloride-activated column is rinsed with 0.1 M Na2CO3 (pH 8.5). For coupling, a peptide or protein (2 mg/ml 0.1 M Na2CO3, pH 8.5) is passed several times for 2 h at 37° C. and then for 4 h on ice through the column. For blocking free binding sites of the column, then an excess of 0.2 M glycine, pH 8 is passed through the column.

### Example 5

### Coupling a Proteoglycan to the Activated Silica Gel Column

The tresyl chloride-activated column is rinsed with 0.1 M Na2CO3 (pH 8.5). For coupling, a peptide or protein (2 mg/ml 0.1 M Na2CO3, pH 8.5) is passed several times over night at room temperature through the column. For blocking free binding sites of the column, then an excess of 0.2 M glycine, pH 8 is passed through the column.

### Example 6

### Coupling a Glycoprotein to the Activated Silica Gel Column

The tresyl chloride-activated column is rinsed with 0.1 M Na2CO3 (pH 8.5). For coupling, a peptide or protein (2 mg/ml 0.1 M Na2CO3, pH 8.5) is passed several times for 2 h at 37° C. and then for 4 h on ice through the column. For blocking free binding sites of the column, then an excess of 0.2 M glycine, pH 8 is passed through the column.

### Example 7

### Coupling a Glycoprotein to the Activated Polyethylene Column

The activated column is rinsed with 0.1 M Na2CO3 (pH 8.5). For coupling, a peptide or protein (2 mg/ml 0.1 M Na2CO3, pH 8.5) is passed several times for 2 h at 37° C. and then for 4 h on ice through the column. For blocking free binding sites of the column, then an excess of 0.2 M glycine, pH 8 is passed through the column. For improving the reaction, 1-ethyl-3-(3-dimethylaminopropyl) carbodiimide (EDC), 5% w/v may be added.

### Example 8

### Preparing a Column for the Elimination of Undesired Molecules

The tresyl chloride-activated column is rinsed with 0.1 M Na2CO3 (pH 8.5). If the elimination is intended against a molecule with one or several primary or secondary amines, this molecule or the mixture (2 mg/ml 0.1 M Na2CO3, pH 8.5) is passed several times over night at room temperature through the column. For blocking free binding sites of the column, then an excess of 0.2 M glycine, pH 8 is passed through the column. If no elimination against certain molecules with one or several primary or secondary amines is desired, all binding sites of the column with glycine are blocked.

Further derivatization methods can for instance be found in the following documents: Patterson, W. J., National Aeronautics and Space Administration, technical Memorandum, NASA TM-73311, U.S. Government Printing Office, Washington, D.C., 1976, Ma, S. M., Gregonis, D. E., van Wagenen, R. M., and Andrade, J. D., in “Hydro gels for Medical and Related Applications” (J. D. Andrade, Ed.), Amer. Chem. Soc. Symp. Series, Vol. 31, p. 241, 1976, Harris, J. M., Struck, E. C., Case, M. G., Paley, M. S., Van Alstine, J. M., and Brooks, D. E., J. Polymer Sci., Polymer Chem. Ed., 22, 341, (1984), Regnier and Noel, Regnier, F. E., and Noel, R. J., J. Chromatog. Sci., 14 (1976), Yalpani, M. and Brooks, D. E., J. Polymer Sci., Polymer Chem. Ed., 23, 395 (1985).

### Example 9

### Carrying-Out the Contact of Nucleic Acids to Target Molecules and Separating the Column

The coated columns are arranged in a leakage-free manner one behind the other, first the columns for the elimination of undesired molecules, then the column with the target molecules. It is possible to also link columns with respectively different target molecules in a leakage-free manner one behind the other, since after removal of nucleic acids with undesired binding properties from the nucleic acid mixture and removal of nucleic acids with binding specificity for one or several target molecules due to the combinatorial composition nucleic acids against a multitude of further target molecules are present. For the equilibration, a suitable buffer, e.g. a buffer solution (10 mM Tris-HCl, 50 mM KCl, 1.5 mM MgCl2 and gelatin 0.001% (w/v), pH 8.3), is passed for 1 h on ice over the column. The nucleic acids of the combinatorial nucleic acid library are received in 1 ml of the same buffer and heated for 10 min to 95° C. for melting the double strands and are then passed for several times (4 to 30 times) over the column. Then the columns are separated and washed over night on ice with the selected buffer (see above). The separation of the column in the direction of flow is effected with a suitable cutting tool.

1. A method for selecting nucleic acids that bond with high affinity to a target molecule from a mixture of nucleic acids, comprising the following steps:
a) a column is loaded with the target molecules and the target molecules are immobilized in said column,
b) the mixture of nucleic acids is fed to a first end of the column, wherein a defined volumetric flow of medium through the column is created, running from the first end to the second end of said column,
c) the nucleic acids bond with an affinity to the target molecule that decreases as the distance from the first end of the column increases and are immobilized,
d) the volumetric flow of medium through the column is stopped after a defined period of time,
e) the column is divided into column segments by a plurality of partitions, a routing co-ordinate being allocated to each segment,
f) the immobilized nucleic acids from at least one segment are desorbed in a non-specific manner and are extracted wherein the routing co-ordinate allocated to the segment are allocated to the desorbed nucleic acids.

2. The method according to claim 1, wherein in step f) the immobilized nucleic acids from each segment are separately desorbed and obtained under respective allocation of the routing co-ordinate of each segment to the nucleic acids obtained therefrom.

3. The method according to claim 1, wherein the non-specific desorption is performed using physico-chemical or thermal methods.

4. The method according to claim 1, wherein the non-specific desorption is performed by thermal desorption in an extended high-temperature phase of an amplification, comprising PCR or RT-PCR.

5. The method according to claim 1, wherein the non-specific desorption is performed or promoted by a chemical modification of the target molecule or by non-specific chelation of the target molecule with a chelating agent.

6. The method according to claim 1, wherein between steps d) and e) at least one washing step is performed.

7. The method according to claim 1, wherein an inner-side loading of the column with target molecules is performed by means of covalent binding, comprising bifunctional spacer compounds.

8. The method according to claim 1, wherein each column segment contains in a statistical average 0.1 to 10³ target molecules.

9. The method according to claim 1, wherein as the structural material of the column, derivatized of plasma-activated silica gel or polyethylene.

10. The method according to claim 1, wherein the length of the column segments is in the range from 0.1 μm to 1 mm.

11. The method according to claim 1, wherein the inner diameter of the column is in the range from 0.05 to 1 mm.

12. Nucleic acids or nucleic acid mixtures obtainable by a method according to claim 1.
